(19) United States
(12) Patent Application Publication
(10) Pub. No.: US 2013/0211370 A1
(43) Pub. Date: Aug. 15, 2013

(54) Medicine Delivery Device

(71) Applicants: Philippe Nzike, Frankfurt am Main (DE); Michael Sachabbach, Frankfurt am Main (DE)

(72) Inventors: Philippe Nzike, Frankfurt am Main (DE); Michael Sachabbach, Frankfurt am Main (DE)

(73) Assignee: Sanofi-Aventis Deutschland GmbH, Frankfurt am Main (DE)

(21) Appl. No.: 13/880,763

(22) PCT Filed: Oct. 21, 2011
(86) PCT No.: PCT/EP2011/068388

§ 371 Date: Apr. 22, 2013

(30) Foreign Application Priority Data
Oct. 25, 2010 (EP) 10188745.3

(51) Int. Cl. A61M 5/145

(52) U.S. Cl. CPC A61M 5/1452
USPC 604/500; 604/152

(57) ABSTRACT

The invention relates to an apparatus comprising a first piezoelectric actuator comprising a shaft, the first piezoelectric actuator being configured to generate a reciprocating movement of the shaft for rotating a piston at least unidirectionally; the piston comprising a stopper being configured to deliver a medicine from a medicine container; and a clutch in contact with the piston; the clutch being configured to translate the rotational movement of the piston into a translational movement of the piston in a forward direction for delivering the medicine from the medicine container; and the clutch being configured to enable a backward movement of the piston in case a force applied to the piston in a backward direction exceeds a predefined value. The invention further relates to a method for delivering medicine and enabling refilling of a medicine container of the apparatus.

### CROSS REFERENCE TO RELATED APPLICATIONS

The present application is a U.S. National Phase Application pursuant to 35 U.S.C. §371 of International Application No. PCT/EP2011/068388 filed Oct. 21, 2011, which claims priority to European Patent Application No. 10188745.3 filed Oct. 25, 2010. The entire disclosure contents of these applications are herewith incorporated by reference into the present application.

### FIELD OF INVENTION

The present invention relates to an apparatus and a method for delivering medicine and enabling refilling of a medicine container of the apparatus.

### BACKGROUND

Nowadays, several medicine devices, such as injection devices, infusion devices, or the like, comprise medicine delivery systems, in particular, pump systems configured to deliver liquid medicines and/or powdery medicines. In general, these devices comprise an energy source, a controller, and an electric motor for actuating a piston to deliver the medicine from a removable or fixed container. The piston may comprise a suitable stopper for delivering the medicine from the medicine container formed cylindrically. Furthermore, it may be possible that the medicine device comprise a needle and a respective needle system for expelling the medicament, for example, for leading the medicine directly into the skin of a patient.

As an electrical motor for instance a stepper motor or a piezoelectric motor can be employed. A piezoelectric motor comprises generally piezoelectric elements which perform a mechanical change, in particular, a change in length depending on an electrical voltage. In case, an alternate voltage is applied the piezoelectric elements vibrates and can generate a reciprocating movement of a connected shaft of the piezoelectric actuator.

German patent application publication 10 2005 004 498 discloses a drive for medicine devices, such as a liquid medicine delivery device. The drive comprises a piezoelectric actuator having a shaft. One end of the shaft is connected to piezoelectric elements while the other end is in contact with a ratchet wheel. The piezoelectric actuator produces a reciprocating linear movement of the shaft. This movement is used to turn the wheel. For enabling a movement of the wheel merely in one direction there is provided a pawl. The pawl is configured such that a movement of the wheel due to any force applied to the wheel in the locked direction is prevented. A disadvantage of the medicine device for delivering medicine is that as a medicine container merely changeable container are used.

International patent application publication 2009/049894 discloses a medicine delivery pump drive using a linear piezoelectric motor. The piezoelectric motor has a shaft and is configured to produce a reciprocating linear movement of the shaft. Furthermore, the medicine delivery pump drive includes a pawl operably connected to the shaft and engaging the ratchet wheel such that the reciprocating linear movement of the shaft is translated into unidirectional rotary movement of the ratchet wheel about the rotational axis which moves the lead screw and advances the piston to deliver the liquid medicine from the medicine container.

A disadvantage of this prior art device is that resetting the apparatus, in particular, the piston to an initial position, for instance, to refill the container, is cumbersome. The pawl must be reset manually.

### SUMMARY

It is thus inter alia an object of the present invention to provide an apparatus and a method which enable to refill an apparatus easily, and at the same time, prevent undesired resetting of the apparatus.

According to a first aspect of the present invention, an apparatus is disclosed, comprising a first piezoelectric actuator comprising a shaft, the first piezoelectric actuator being configured to generate a reciprocating movement of the shaft for rotating a piston at least unidirectionally; the piston comprising a stopper being configured to deliver a medicine from a medicine container; a clutch in contact with the piston; the clutch being configured to translate the rotational movement of the piston into a translational movement of the piston in a forward direction for delivering the medicine from the medicine container; and the clutch being configured to enable a backward movement of the piston in case a force applied to the piston in a backward direction exceeds a predefined value.

According to a second aspect of the present invention, a method is disclosed, comprising generating a reciprocating movement of the shaft by a first piezoelectric actuator; rotating a piston by the reciprocating movement of the shaft; translating the rotational movement of the piston into a translational movement of the piston in forward direction for delivering a medicine from a medicine container; and refilling the medicine container by moving the piston backward in case a force applied to the piston in a backward direction exceeds a predefined value.

Accordingly, also an apparatus configured to perform the method according to the second aspect of the present invention shall be considered to be disclosed.

In the following, features and embodiments (exhibiting further features) of the present invention will be described, which are understood to equally apply to the apparatus and the method described above. In particular, a mentioning that a component is configured or arranged to perform a certain action should be understood to also disclose an according method step of the method according to the second aspect of the present invention. These single features/embodiments are considered to be exemplary and non-limiting, and to be respectively combinable independently from other disclosed features/embodiments with the apparatus and method of the present invention as described above. Nevertheless, these features/embodiments shall also be considered to be disclosed in all possible combination with each other and with the apparatus and method of the present invention as described above.

The apparatus, according to the first aspect of the present invention, for instance a medicine delivery device, like a pump device, comprises as an electrical motor at least one piezoelectric actuator. A piezoelectric actuator has the advantage of low power consumption and the possibility to drive a piston with high precision. The piezoelectric actuator may comprise a shaft for transmitting a reciprocating movement generated by piezoelectric elements to a piston. A reciprocating movement can be divided into a first movement from a starting point to an intermediate point and a second movement from the intermediate point to the starting point. The reciprocating movement of the shaft is translated into a unidirectional rotary movement of the piston.

The piston comprises a suitable stopper arranged at one end of the piston and integrated in a medicine container. Generally, a forward movement of the piston causes the medicine to deliver from the container in a controlled manner. By way of example, medicine doses in the range of 100 nl can be delivered accurately.

More particularly, the rotational movement of the piston generated by the piezoelectric actuator is translated in a respective translational movement in the forward direction by a clutch. In other words, the clutch generates a forward movement of the piston depending on the unidirectional rotary movement of the piston to deliver medicine from the container in a controlled manner.

In case all or part of the medicine is delivered from the container refilling of the container may be required. For enabling refilling of the container for instance by a patient in an easy manner, the clutch is configured to enable a backward movement of the piston if a force applied to the piston in a backward direction exceeds a predefined value. For instance, new medicine may be pushed into the container generating a force applied to the piston, in particular, the stopper of the piston in an opposite delivering direction. If the force is equal or larger than the predefined value the clutch is configured such that the piston is moved backward until the applied force falls below the predefined value or the initial position of the piston is reached, i.e. the container is totally refilled.

Thereby, the predefined value can be defined such that an undesired backward movement of the piston is securely prevented. For instance, the pressure onto the stopper in opposite direction can increase due to temperature change, position change of the apparatus or the like. The predefined value is set such that the forces generated through the previously described events are smaller than the predefined value for avoiding an undesired backward movement of the piston. At the same time, the predefined value can be defined that refilling is enabled for a patient in an easy manner. For instance, the patient may use merely his/her normal muscular strength instead of a complex mechanism.

According to an exemplified embodiment of the invention, the apparatus may further comprise a wheel connected to the piston configured to translate a movement of the wheel to a corresponding movement of the piston, wherein the shaft may be configured to rotate the piston via the wheel. For instance, the shaft can contact the wheel during the first movement and/or the second movement for generating a unidirectional rotary movement of the wheel. The piston and the wheel may be coupled such that the wheel can merely perform a rotary movement while the piston may perform a rotary movement and a translational movement. Implementing a wheel for translating the force generated by the at least one piezoelectric actuator to the rotary movement of the piston results in reduced friction losses.

Generally, the first piezoelectric actuator may be configured to perform a linear or a non-linear reciprocating movement. In case a linear reciprocating movement is generated, the shaft may contact the wheel during both movements. A return movement can be prevented by employing an additional pawl. According to another embodiment of the present invention, the first piezoelectric actuator may be configured to generate a non-linear reciprocating movement of the shaft for contacting the wheel during one movement of the reciprocating movement of the shaft. In other words, the shaft may perform such a movement that it drives the wheel and piston, respectively, in merely one direction. For instance, the shaft may be in contact with the wheel during the first movement only. An example of a non-linear reciprocating movement is a movement in an elliptical form. Such an elliptical reciprocating movement can be achieved by generating a respective change in length of the shaft. The change in length of the shaft can be created by piezoelectric elements.

For producing a formfitting contact between a tip of the shaft and the wheel for rotating the wheel by the shaft, according to an embodiment of the present invention, the first piezoelectric actuator may further comprise a first resetting element. The restoring force of the resetting element may push the shaft tip at least during one movement of the reciprocating movement onto the wheel such that the shaft moves the wheel in a controlled and accurate manner.

A particular good contact between the shaft and the wheel can be achieved in case the tip of the shaft comprises a dented shape.

According to another embodiment of the present invention the clutch may be formed as a slipping clutch. A slipping clutch is a force fit and self switching clutch.

For guiding the piston, according to an embodiment of the present invention, the clutch may comprise at least two pins configured to guide the piston. Guiding is to be understood that a controlled movement of the piston in a longitudinal direction of the container is provided for. For example, the pins can be fixedly connected to the chassis of the apparatus and arranged on both sides of the piston. In other words, the piston may be fixed between the at least two pins. Preferably, the pins may comprise a cylindrical shape.

According to a further exemplified embodiment, each pin may comprise a movable pinion having a thread corresponding to a thread of the piston and a blocking element configured to prevent a movement of the pinion such that the rotational movement of the piston is translated into a translational movement of the piston in forward direction. Although the pinion may be generally moveable in a rotary and/or longitudinal direction of a cylindrical pin, the blocking element can be formed such that during the rotary movement of the shaft the pinion cannot be moved. The thread of the piston and the threads of the pinions may cooperate such that the desired forward movement for delivering the medicine in a controlled manner can be achieved depending on the movement of the shaft.

According to an exemplified embodiment, the backward movement of the piston can be achieved by allowing a movement of the pinion depending on predefined conditions. For instance, the clutch may be configured to enable a movement of the pinion such that a backward movement of the piston is enabled in case the applied force exceeds the predefined value. By way of example, the applied force causes a rotary movement of the pinions resulting in that the piston is moved backward, especially, slipped backward due to the thread connection between the pinions and the piston.

Furthermore, each pin may comprise a second resetting element having a restoring force, wherein the predefined value depends on the restoring force of the second resetting element. For instance, the predefined value may be identical with the value of the restoring forces.

The second resetting element may be formed as a spring. A spring is particular suitable as a resetting element. A spring comprises a spring force which may define the predefined value. Therefore, the predefined value can be defined in an easy manner by implementing respective springs. If the applied force exceeds the spring forces, the piston may be moved backward.

According to another embodiment of the present invention, the apparatus may comprise a medicine delivery opening for delivering the medicine, wherein the medicine delivery opening may correspond to an opening of a refill package. In other words, merely one opening is provided to deliver and refill the container of the apparatus. It shall be understood that according to other variants of the present invention the apparatus may comprise also an additional refill opening. For instance, a patient may insert the opening of the refill package, like a suitable plastic bottle, into the corresponding opening of the apparatus and may refill the container by causing the medicine to dispense from the bottle. For instance, the bottle may comprise a suitable mechanism, like a spray head, which allows dispensing the medicine in an automatic or manual manner.

To improve the performance of the apparatus, according to an embodiment of the present invention, the apparatus may comprise at least one further piezoelectric actuator. The employment of two or more piezoelectric actuators may allow increasing the available force transmitted to the piston.

In general, the apparatus may be used for delivering powdery medicines and/or liquid medicines. According to an embodiment, the apparatus may be a liquid medicine delivery device.

The apparatus may be an injection device. It may for instance be an injection device for injecting fluids into material, e.g. a body of a creature, wherein the injection is executed with the injection device by an entity (a human being or a machine). The injected fluid may for instance be a drug, such as for instance insulin. The human being executing the injection may then for instance be the patient receiving the injection, or another person.

According to a further embodiment of the present invention, the apparatus may be an insulin injector. An insulin injector is an example of a device configured to administer a medicament, i.e. insulin, to a patient. An insulin injector may for instance be embodied as an insulin pump.

According to another embodiment of the present invention, the apparatus may be an infusion device. An infusion device is an example of a device configured to administer a liquid substance, for instance crystalloids or colloids, to a patient. An infusion device may for instance be embodied as an infusion pump.

According to a further embodiment of the present invention, the apparatus may comprise an energy source configured to supply the apparatus with energy. For instance, the energy source may be any kind of battery and/or a photovoltaic cell.

Furthermore, according to an embodiment of the present invention, the apparatus may further comprise a controller configured to control the operation of the apparatus. The processor of the apparatus may for instance be embodied as a microprocessor, a Digital Signal Processor (DSP), Application Specific Integrated Circuit (ASIC), Field Programmable Gate Array (FPGA) or the like.

The apparatus according another embodiment may comprise at least one sensor configured to monitor the operation of the apparatus. By way of example, a filling sensor configured to measure the fill level of the medicine in the container and/or a rotational sensor configured to measure the actual position of the wheel and piston, respectively, can be employed.

According to another embodiment of the present invention, the apparatus may comprise a bearing configured to fix the piston to a chassis of the apparatus.

Furthermore, according to an embodiment of the present invention, the apparatus may further comprise an abdomen belt configured to fix the apparatus around the abdomen of a patient.

Furthermore, according to an embodiment of the present invention, software adapted to control the piezoelectric actuator may be provided.

It is to be understood that the features of any presented embodiment can be combined with the features of any other embodiment.

### BRIEF DESCRIPTION OF THE DRAWINGS

Exemplary embodiments of the invention will be described in more detail in the following with reference to drawings.

In the figures show:

FIG. 1 a schematic illustration of an apparatus according to a first embodiment of the present invention;

FIG. 2 a schematic illustration of a piezoelectric actuator according to an embodiment of the present invention;

FIG. 3 a further schematic illustration of a piezoelectric actuator according to an embodiment of the present invention;

FIG. 4 a first schematic partial illustration of an apparatus according to the first embodiment of the present invention;

FIG. 5 a schematic sectional view of the partial illustration of an apparatus according to FIG. 4;

FIG. 6 a second schematic partial illustration of an apparatus according to a first embodiment of the present invention;

FIG. 7 a schematic sectional view of the partial illustration of an apparatus according to FIG. 6; and

FIG. 8 a flowchart of a method according to an embodiment of the present invention.

### DETAILED DESCRIPTION

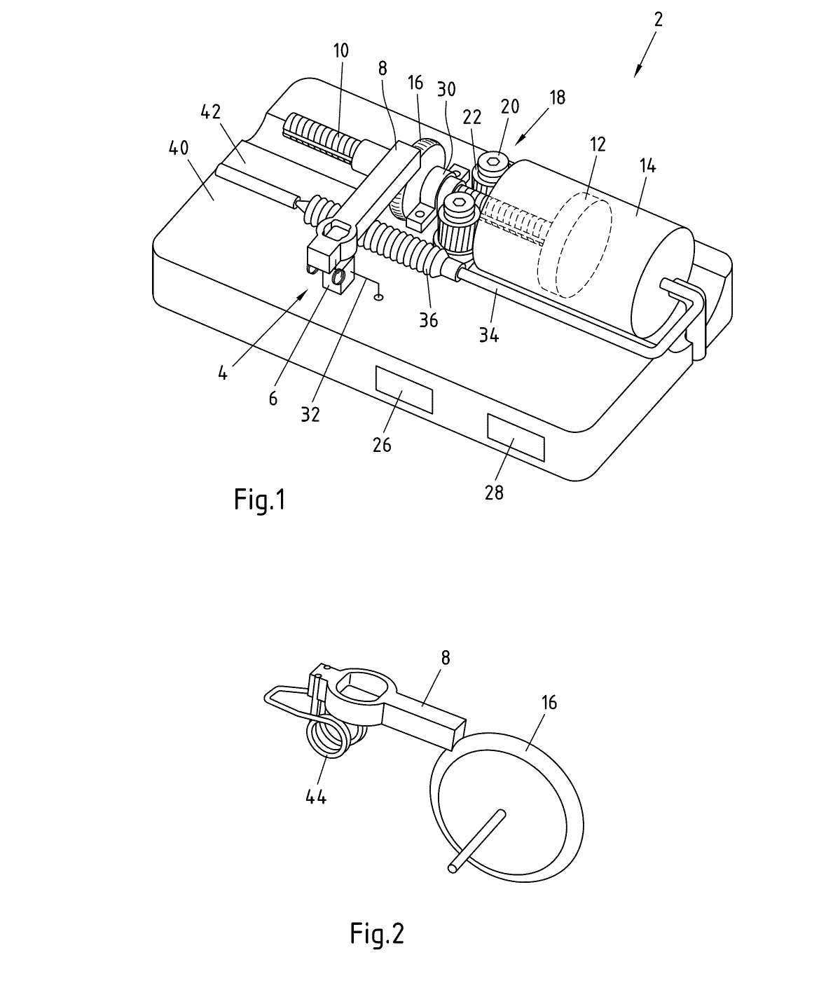

FIG. 1 is a schematic illustration of an apparatus according to a first embodiment of the present invention. The depicted exemplary apparatus 2 is a liquid medicine delivery device 2. It shall be understood that according to other variants of the present invention the apparatus may be also a delivery device for powdery medicines.

For instance, the liquid medicine delivery device 2 can be an insulin injector or another medicament injector.

The liquid medicine delivery device 2 may comprise an energy source 28, like a battery 28. The battery 28 can be exchangeable or non-exchangeable. It can further be rechargeable or non-rechargeable.

The battery 28 is arranged to supply energy to a processor 26 and a piezoelectric actuator 4. The processor 26 may for instance be a microprocessor, a Digital Signal Processor (DSP), an Application Specific Integrated Circuit (ASIC), a Field Programmable Gate Array (FPGA) or the like. Processor 26 may be configured to control the piezoelectric actuator 4.

Furthermore, wirings 32 are provided for supplying and controlling the piezoelectric actuator 4. The piezoelectric actuator 4 may comprise a piezoelectric element 6 configured to produce the piezoelectric effect. Further it may comprise a shaft 8.

The piezoelectric actuator 4 may be configured to rotate a piston 10 via a wheel 16 wherein the piston 10 and the wheel 16 are connected to each other. The piston 10 and the wheel 16 may be coupled such that the wheel 16 can merely perform a rotary movement while the piston 10 may perform a rotary movement and a translational movement. As can be seen from FIG. 1, the piston 10 comprises a longitudinal groove to allow a forward movement of the piston 10 for delivering the medicine. The wheel 16 has a corresponding groove for allowing this forward movement of the piston 10 independent of the rotary wheel movement. To fix the piston 10 to the chassis of the apparatus 2, there may be provided a bearing 30.

According to the exemplified embodiment, the piston 10 comprises a stopper 12 and may be guided by a clutch 18. The clutch 18 may be a slipping clutch. It may comprise two pins, each having a second resetting element 20, a pinion 22 and a blocking element 24. The pins can be fixedly connected to the chassis of the apparatus 2 and arranged on both sides of the piston 10 to fix the piston 10 between them. As can be seen, the pins may comprise a cylindrical shape.

Additionally, a medicine container 14 may be arranged. In general, the medicine container 14 may be changeable or non-changeable. A changeable container 14 may or may not comprise the piston 10. In case the piston 10 is a fixed part of the apparatus 2 and not exchangeable, inserting the new container 14 comprises connecting the piston 10 to the container 14 fixedly. If the piston 10 belongs to the exchangeable medicine container 14, inserting the container with the piston 10 can be performed in an analogous way to the refilling method, as described in detail hereinafter.

In the present exemplified embodiment the container 14 is a non-changeable medicine container 14 configured to store the medicine to be delivered. The medicine may be for instance insulin, an infusion or the like.

The medicine may be delivered via tube 34 and 42 by a needle (not shown) into the skin of a patient. Furthermore, reference sign 32 indicates a plastic protection configured to protect the needle and reference sign 42 indicates a needle support.

In the following the piezoelectric actuator 4 and its functioning will be elucidated in more detail.

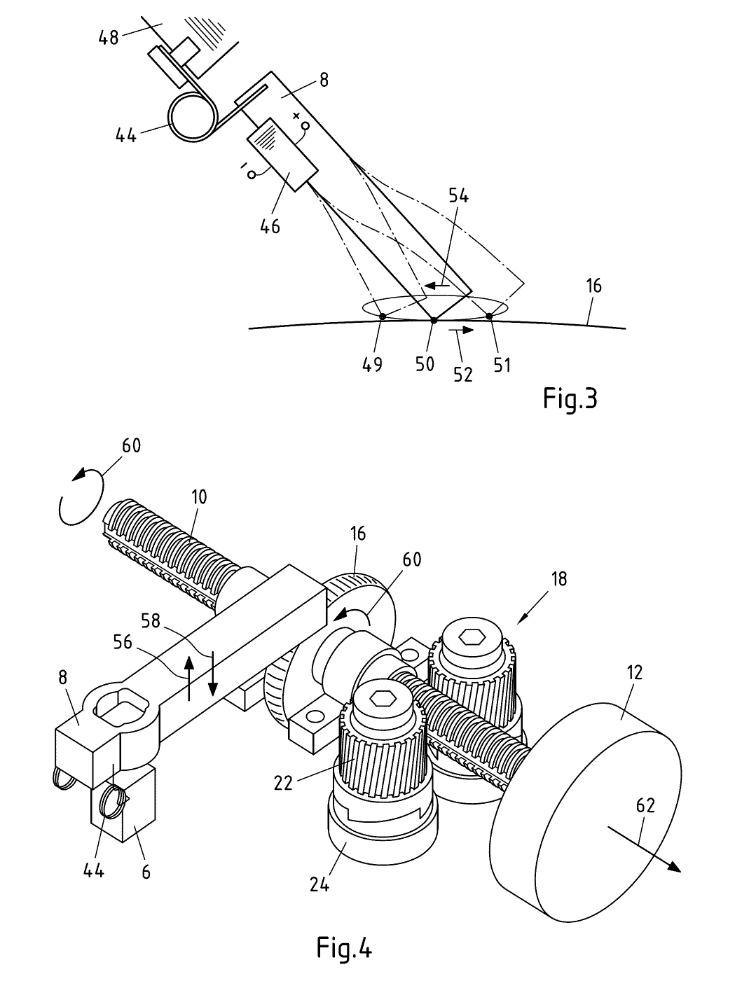

FIGS. 2 and 3 show the piezoelectric actuator 4 according to an embodiment of the present invention in more detail. Besides the shaft 8 the actuator 4 may comprise a first resetting element 44. The first resetting element 44 may be a spring having a respective spring force. The first resetting element 44 may be configured to produce a formfitting contact between a tip of the shaft 8 and the wheel 16. For producing a particular good contact with the wheel 16 the shaft tip may have a dented shape.

The shaft 8 may be connected to a mounting 48 via the spring 44. To move the wheel 16 unidirectionally, the exemplified actuator is implemented as follows. By applying a voltage to the shaft 8, in particular, an alternate voltage, a change in length of the shaft 8 can be achieved. The change in length of the shaft 8 causes that a non-linear reciprocating movement can be produced. In particular, an elliptic movement can be generated, as depicted in FIG. 3. A first movement 52 from a starting point 49 to an intermediate point 51 may be performed via another route than the second movement 54 from the intermediate point 51 to the starting point 49.

During the first movement 50, the tip of the shaft 8 is in contact, in particular, formfitting contact with the surface of the wheel 16. Due to the elliptic movement, the shaft does not contact the wheel 16 during the second movement 52 of the reciprocating movement resulting in that the wheel 16 can be unidirectionally moved some micrometers during each oscillation of the shaft 8. A separate pawl for preventing a return movement of the wheel 16 due to a linear reciprocating movement can be omitted in the present example.

The resulting torque and speed may depend on various parameters, like the wheel diameter. The friction losses may depend on the wheel material, the wheel diameter and the diameter of the piston 10. According to an exemplified embodiment, the diameter of the piston 10 may be no more than one twentieth of the wheel diameter.

Furthermore, the motor power may depend also on various parameters. In particular, the motor power may depend on the rotor material. For achieving a good performance, materials with high stiffness and sufficient stability under heat can be used.

According to an embodiment of the present invention the available power can be increased by employing two or more piezoelectric actuators.

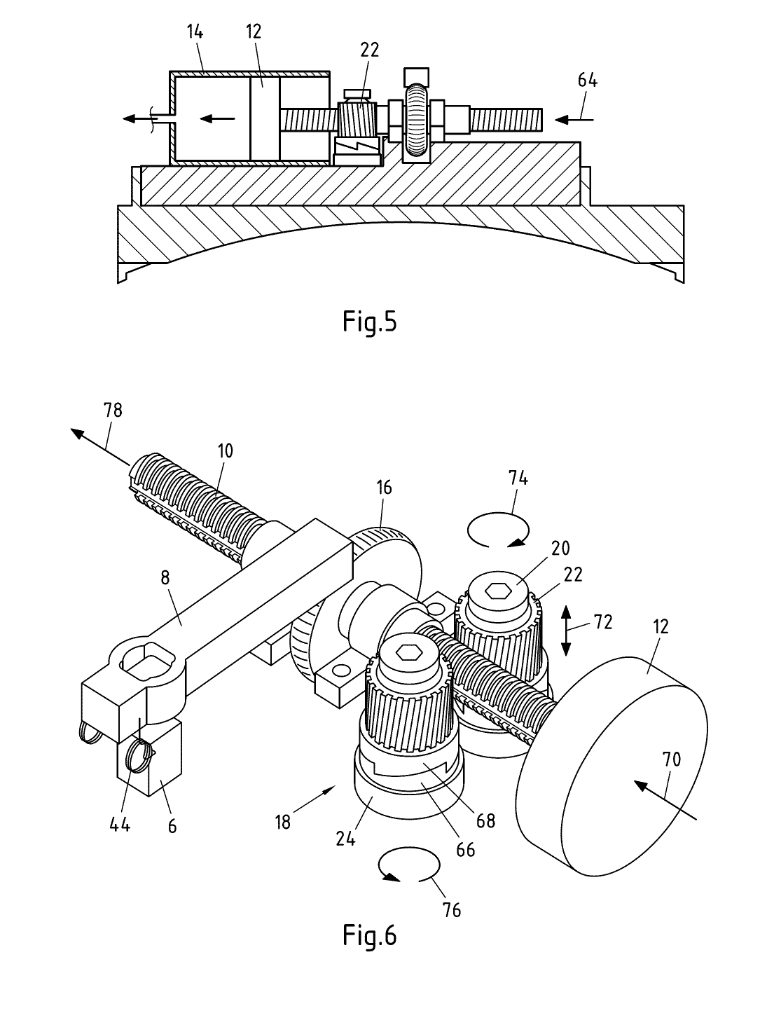

As can be seen from FIGS. 4 and 5, in case the medicine should be delivered from the medicine container 14 the shaft 8 produces a reciprocating movement 56, 58. According to an embodiment, the reciprocating movement comprises an elliptic form resulting in that the wheel 16 is merely rotated in one direction 60. Due to the coupling of wheel 16 and piston 10 the piston 10 rotates also in direction 60.

The clutch 18 is configured such that the rotational movement of the piston 10 is translated into a translational movement, in particular, in forward direction 62, for delivering the medicine.

In the exemplified embodiment of FIG. 4, the clutch 18 comprises two pins, wherein each pin comprises a blocking element 24 and a movable pinion 22. The blocking element 24 prevents a movement of the pinion 22. The thread of the piston 10 and the threads of the pinions 22 enable to translate the rotational movement of the piston 10 into a translational movement of the piston 10 in forward direction 62. The clutch 18 and its functioning will be pointed out in more detail subsequently.

As can be seen from the schematic sectional view of FIG. 5 a forward movement of the piston 10 may cause that the stopper 12 pushes the medicine out of the medicine container 14 in direction 64, for instance, in order to inject the medicine to a patient. In particular, the stopper 12 of the piston 10 may cause the desired medicine dose to deliver from the container 14, wherein a needle may lead the medicine into the skin of the patient.

In the case the medicine container 14 is a non-exchangeable container, the medicine container 14 has to be refilled with the respective medicine from time to time. According to an exemplary embodiment of the invention, the container 14 can be refilled in an easy manner.

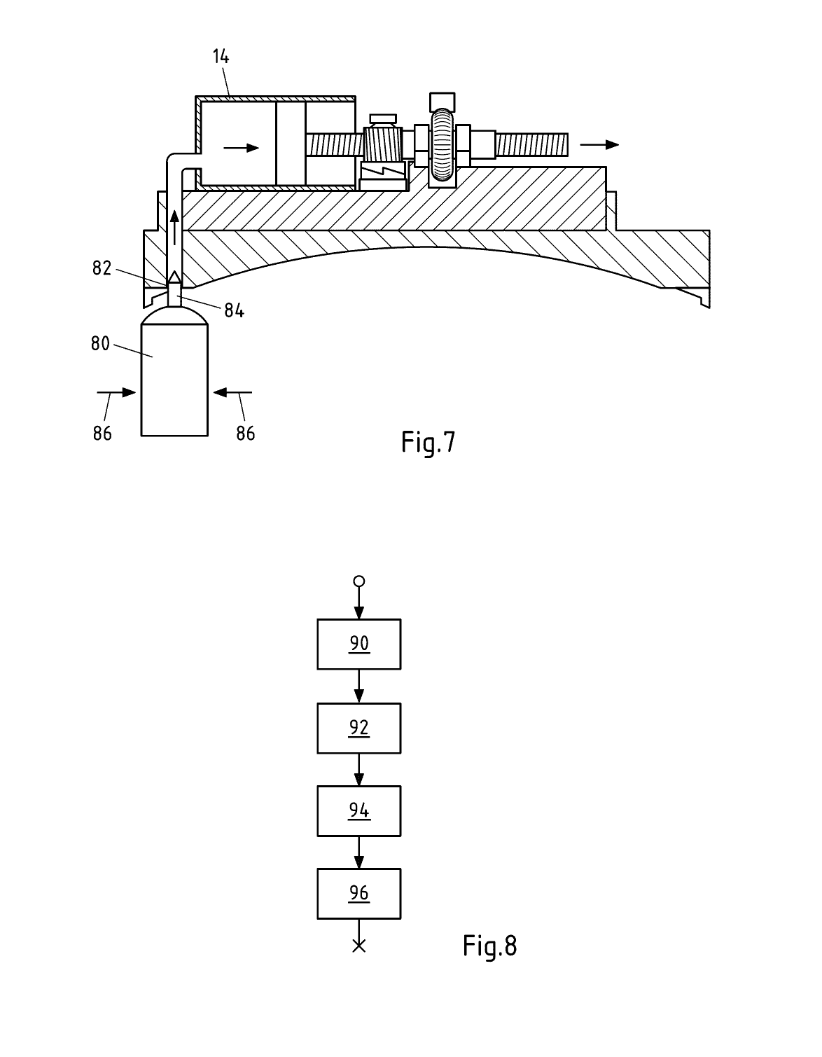

In more detail, as can be seen from FIGS. 6 and 7, the medicine may be provided in a suitable refill package 80, like a plastic bottle 80. The plastic bottle 80 may comprise a suitable opening 84 corresponding to the outlet 82 of the apparatus 2. It shall be understood that according to other variants of the present invention the apparatus may also comprise an additional refill opening.

The patient may insert the opening 84 of the bottle 80 into the corresponding outlet 82 of the apparatus 2 and may cause the medicine to deliver from the bottle 80, for instance, by applying a force 86 to the bottle 80. More particularly, by simply pushing the bottle 80, the medicine may flow into the apparatus 2. It shall be understood that the refill package 80 may comprise other mechanisms for delivering the medicine, for example a syringe like mechanism.

As previously pointed out, the piston 10 may have a thread corresponding to the threads of the pinions 22. If a force is applied to the stopper 12, in particular, due to the flowing medicine in the opposite delivery direction, the stopper 12 may be moved backward due to the design of the clutch 18.

The clutch 18 may be formed as a slipping clutch enabling the piston 10 to move back depending on predefined conditions. In particular, the piston 10 may slip back in case the force applied exceeded a predefined value. For instance, the value can be predefined by a restoring force of a second resetting element 20.

In the following the design and function of an exemplary embodiment of a clutch 18 according to an embodiment of the invention is explained in depth.

The clutch 18 may comprise two pins which may comprise the same design. However, the saw tooth structure 66, 68 is provided in opposite directions. A pin may comprise a base body fixedly connected to the chassis of the apparatus 2. The base body may be formed cylindrically. At the lower end of the base body which is connected to the chassis a circular blocking element 24 may be arranged. An end of the blocking element 24 may be also fixedly connected to the chassis and/or the base body. The other end of the blocking element 24 may comprise a saw tooth shape profile 66, as can be seen from FIGS. 5 and 7.

On top of the blocking element 24 a circular pinion 22 may be disposed. Generally, the pinion 22 may be moveable in the direction of the longitudinal axis and in circumferential direction of the base body. The pinion 22 may also have a saw tooth shape profile 68 corresponding to the saw tooth shape profile 66 of the blocking element 24.

Furthermore, on top of the pinion 22 a second resetting element 20 may be arranged. For instance, this resetting element 20 may be a spring. The spring 20 may be arranged such that it pushes the pinion 22 against the blocking element 24 due to its spring force.

The saw tooth shape profile 66 of the blocking element 24 may be formed such that if the piston 10 is moved by the piezoelectric actuator 4 for delivering medicine a movement of the pinion 22 is prevented since the pinion 22 pushes with its cliff shape of the saw tooth profile 68 against the cliff shape of the saw tooth profile 66 of the blocking element 24. Since the pinion 22 is fixed the rotational movement of the piston 10 can be translated into a translational movement.

For refilling the container 14, as previously described the medicine to be refilled may push against the stopper 12 due to the interaction of a patient. If the force applied to the stopper 12 is at least larger than a predefined value, the piston 10 is moved backward.

More particularly, the predefined value may be sum of the resetting forces of the second resetting elements 20, such as the spring forces. If a force is applied to the stopper 12 in the backward direction which is larger than the spring forces of the springs 20 which push the pinions 22 against the blocking elements 24 the piston 10 transmits the force via the threads to the pinions 22. Then the pinions 22 compress the respective springs 20 and slip over the ramps of the saw tooth profiles 66 of the blocking elements 24. The piston 10 returns back due to the movement of the pinions 22 as long as the applied force is high enough or the initial position of the piston 10 is reached, i.e. the medicine container 14 is completely refilled.

It shall be understood that according to other embodiments of the present invention for refilling the apparatus 2 a new container 14 comprising the piston 10 can be inserted in the same way, as previously described. For instance, after removing the empty medicine container 14, the new medicine container 14 with the piston 10 can be inserted by moving the piston 10 through the clutch 18, the bearing 22 and the wheel 16 in opposite delivering direction.

FIG. 8 shows an exemplified flowchart of the method according to an embodiment of the present invention. In a first step 90, a reciprocating movement of the shaft 8 is generated by a first piezoelectric actuator 4. Then the piston 10 is rotated by the reciprocating movement of the shaft 8 unidirectionally (step 92). This unidirectional rotary movement of the piston 10 is translated into a translational movement of the piston 10 in forward direction for delivering a medicine from a medicine container 14 in a further step 94. In case a refilling of the container 14 is desired, for instance, since the container 14 is empty the container 14 is refilled in the next step 96. A force is applied to a stopper 12 of the piston 10 in a backward direction. If the force exceeds a predefined value the piston 10 is moved backward. Then the method may continue with the first step 90.

The term “medicine” or “medicament”, as used herein, means a pharmaceutical formulation containing at least one pharmaceutically active compound,

wherein in one embodiment the pharmaceutically active compound has a molecular weight up to 1500 Da and/or is a peptide, a proteine, a polysaccharide, a vaccine, a DNA, a RNA, a antibody, an enzyme, an antibody, a hormone or an oligonucleotide, or a mixture of the above-mentioned pharmaceutically active compound,

wherein in a further embodiment the pharmaceutically active compound is useful for the treatment and/or prophylaxis of diabetes mellitus or complications associated with diabetes mellitus such as diabetic retinopathy, thromboembolism disorders such as deep vein or pulmonary thromboembolism, acute coronary syndrome (ACS), angina, myocardial infarction, cancer, macular degeneration, inflammation, hay fever, atherosclerosis and/or rheumatoid arthritis,

wherein in a further embodiment the pharmaceutically active compound comprises at least one peptide for the treatment and/or prophylaxis of diabetes mellitus or complications associated with diabetes mellitus such as diabetic retinopathy,

wherein in a further embodiment the pharmaceutically active compound comprises at least one human insulin or a human insulin analogue or derivative, glucagon-like peptide (GLP-1) or an analogue or derivative thereof, or exedin-3 or exedin-4 or an analogue or derivative of exedin-3 or exedin-4.

Insulin analogues are for example Gly(A21), Arg(B31), Arg(B32) human insulin; Lys(B3), Glu(B29) human insulin; Lys(B28), Pro(B29) human insulin; Asp(B28) human insulin; human insulin, wherein proline in position B28 is replaced by Asp, Lys, Leu, Val or Ala and wherein in position B29 Lys may be replaced by Pro; Ala(B26) human insulin; Des(B28-B30) human insulin; Des(B27) human insulin and Des(B30) human insulin.

Insulin derivates are for example B29-N-myristoyl-des(B30) human insulin; B29-N-palmitoyl-des(B30) human insulin; B29-N-myristoyl human insulin; B29-N-palmitoyl human insulin; B28-N-myristoyl LysB28ProB29 human insulin; B28-N-palmitoyl-LysB28ProB29 human insulin; B30-N-myristoyl-ThrB29LysB30 human insulin; B30-N-palmitoyl-ThrB29LysB30 human insulin; B29-N-(N-palmitoyl-Y-glutamyl)-des(B30) human insulin; B29-N-(N-lithocholyl-Y-glutamyl)-des(B30) human insulin; B29-N-(ω-carboxyheptadecanoyl)-des(B30) human insulin and B29-N-(ω-carboxyhepta-decanoyl) human insulin.

Exendin-4 for example means Exendin-4(1-39), a peptide of the sequence H His-Gly-Glu-Gly-Thr-Phe-Thr-Ser-Asp-Leu-Ser-Lys-Gln-Met-Glu-Glu-Glu-Ala-Val-Arg-Leu-Phe-Ile-Glu-Trp-Leu-Lys-Asn-Gly-Gly-Pro-Ser-Ser-Gly-Ala-Pro-Pro-Pro-Ser-NH2.

Exendin-4 derivatives are for example selected from the following list of compounds:

H-(Lys)4-des Pro36, des Pro37 Exendin-4(1-39)-NH2,

H-(Lys)5-des Pro36, des Pro37 Exendin-4(1-39)-NH2,

des Pro36 [Asp28] Exendin-4(1-39),

des Pro36 [IsoAsp28] Exendin-4(1-39),

des Pro36 [Met(0)14, Asp28] Exendin-4(1-39),

des Pro36 [Met(O)14, IsoAsp28] Exendin-4(1-39),

des Pro36 [Trp(O2)25, Asp28] Exendin-4(1-39),

des Pro36 [Trp(O2)25, IsoAsp28] Exendin-4(1-39),

des Pro36 [Met(O)14 Trp(O2)25, Asp28] Exendin-4(1-39),

des Pro36 [Met(O)14 Trp(O2)25, IsoAsp28] Exendin-4(1-39); or

des Pro36 [Asp28] Exendin-4(1-39),

des Pro36 [IsoAsp28] Exendin-4(1-39),

des Pro36 [Met(O)14, Asp28] Exendin-4(1-39),

des Pro36 [Met(O)14, IsoAsp28] Exendin-4(1-39),

des Pro36 [Trp(O2)25, Asp28] Exendin-4(1-39),

des Pro36 [Trp(O2)25, IsoAsp28] Exendin-4(1-39),

des Pro36 [Met(O)14 Trp(O2)25, Asp28] Exendin-4(1-39),

des Pro36 [Met(O)14 Trp(O2)25, IsoAsp28] Exendin-4(1-39),

wherein the group -Lys6-NH2 may be bound to the C-terminus of the Exendin-4 derivative;

or an Exendin-4 derivative of the sequence

H-(Lys)6-des Pro36 [Asp28] Exendin-4(1-39)-Lys6-NH2,

des Asp28 Pro36, Pro37, Pro38Exendin-4(1-39)-NH2,

H-(Lys)6-des Pro36, Pro38 [Asp28] Exendin-4(1-39)-NH2,

H-Asn-(Glu)5des Pro36, Pro37, Pro38 [Asp28] Exendin-4(1-39)-NH2,

des Pro36, Pro37, Pro38 [Asp28] Exendin-4(1-39)-(Lys)6-NH2,

H-(Lys)6-des Pro36, Pro37, Pro38 [Asp28] Exendin-4(1-39)-(Lys)6-NH2,

H-Asn-(Glu)5-des Pro36, Pro37, Pro38 [Asp28] Exendin-4(1-39)-(Lys)6-NH2,

H-(Lys)6-des Pro36 [Trp(O2)25, Asp28] Exendin-4(1-39)-Lys6-NH2,

H-des Asp28 Pro36, Pro37, Pro38 [Trp(O2)25] Exendin-4(1-39)-NH2,

H-(Lys)6-des Pro36, Pro37, Pro38 [Trp(O2)25, Asp28] Exendin-4(1-39)-NH2,

H-Asn-(Glu)5-des Pro36, Pro37, Pro38 [Trp(O2)25, Asp28] Exendin-4(1-39)-NH2,

des Pro36, Pro37, Pro38 [Trp(O2)25, Asp28] Exendin-4(1-39)-(Lys)6-NH2,

H-(Lys)6-des Pro36, Pro37, Pro38 [Trp(O2)25, Asp28] Exendin-4(1-39)-(Lys)6-NH2,

H-Asn-(Glu)5-des Pro36, Pro37, Pro38 [Trp(O2)25, Asp28] Exendin-4(1-39)-(Lys)6-NH2,

H-(Lys)6-des Pro36 [Met(O)14, Asp28] Exendin-4(1-39)-Lys6-NH2,

des Met(O)14 Asp28 Pro36, Pro37, Pro38 Exendin-4(1-39)-NH2,

H-(Lys)6-desPro36, Pro37, Pro38 [Met(O)14, Asp28] Exendin-4(1-39)-NH2,

H-Asn-(Glu)5-des Pro36, Pro37, Pro38 [Met(O)14, Asp28] Exendin-4(1-39)-NH2,

des Pro36, Pro37, Pro38 [Met(O)14, Asp28] Exendin-4(1-39)-(Lys)6-NH2,

H-(Lys)6-des Pro36, Pro37, Pro38 [Met(O)14, Asp28] Exendin-4(1-39)-(Lys)6-NH2,

H-Asn-(Glu)5 des Pro36, Pro37, Pro38 [Met(O)14, Asp28] Exendin-4(1-39)-(Lys)6-NH2,

H-Lys6-des Pro36 [Met(O)14, Trp(O2)25, Asp28] Exendin-4(1-39)-Lys6-NH2,

H-des Asp28 Pro36, Pro37, Pro38 [Met(O)14, Trp(O2)25] Exendin-4(1-39)-NH2,

H-(Lys)6-des Pro36, Pro37, Pro38 [Met(O)14, Asp28] Exendin-4(1-39)-NH2,

H-Asn-(Glu)5-des Pro36, Pro37, Pro38 [Met(O)14, Trp(O2)25, Asp28] Exendin-4(1-39)-NH2,

des Pro36, Pro37, Pro38 [Met(O)14, Trp(O2)25, Asp28] Exendin-4(1-39)-(Lys)6-NH2,

H-(Lys)6-des Pro36, Pro37, Pro38 [Met(O)14, Trp(O2)25, Asp28] Exendin-4(S1-39)-(Lys)6-NH2,

H-Asn-(Glu)5-des Pro36, Pro37, Pro38 [Met(O)14, Trp(O2)25, Asp28] Exendin-4(1-39)-(Lys)6-NH2;

or a pharmaceutically acceptable salt or solvate of any one of the afore-mentioned Exedin-4 derivative.

Hormones are for example hypophysis hormones or hypothalamus hormones or regulatory active peptides and their antagonists as listed in Rote Liste, ed. 2008, Chapter 50, such as Gonadotropine (Follitropin, Lutropin, Choriongonadotropin, Menotropin), Somatropine (Somatropin), Desmopressin, Terlipressin, Gonadorelin, Triptorelin, Leuprorelin, Buserelin, Nafarelin, Goserelin.

A polysaccharide is for example a glucosaminoglycane, a hyaluronic acid, a heparin, a low molecular weight heparin or an ultra low molecular weight heparin or a derivative thereof, or a sulphated, e.g. a poly-sulphated form of the above-mentioned polysaccharides, and/or a pharmaceutically acceptable salt thereof. An example of a pharmaceutically acceptable salt of a poly-sulphated low molecular weight heparin is enoxaparin sodium.

Pharmaceutically acceptable salts are for example acid addition salts and basic salts. Acid addition salts are e.g. HCl or HBr salts. Basic salts are e.g. salts having a cation selected from alkali or alkaline, e.g. Na+, or K+, or Ca2+, or an ammonium ion N+(R1)(R2)(R3)(R4), wherein R1 to R4 independently of each other mean: hydrogen, an optionally substituted C1 C6-alkyl group, an optionally substituted C2-C6-alkenyl group, an optionally substituted C6-C10-aryl group, or an optionally substituted C6-C10-heteroaryl group. Further examples of pharmaceutically acceptable salts are described in “Remington's Pharmaceutical Sciences” 17. ed. Alfonso R. Gennaro (Ed.), Mark Publishing Company, Easton, Pa., U.S.A., 1985 and in Encyclopedia of Pharmaceutical Technology.

Pharmaceutically acceptable solvates are for example hydrates.

The invention has been described above by means of embodiments, which shall be understood to be non-limiting examples only. In particular, it should be noted that there are alternative ways and variations which are obvious to a skilled person in the art and can be implemented without deviating from the scope and spirit of the appended claims.

It should also be understood that the sequence of method steps on the flowchart presented above is not mandatory. Alternative sequences may be possible. All functional blocks of apparatuses shall also be understood as a disclosure of an according method step, and similarly, each method step shall be considered as a disclosure of an according functional unit of an apparatus. It is well understood that the method steps and functional components can be implemented in various ways either in hardware only or in software only, or in a combination of hard- and software.

1. An apparatus comprising:
a piston comprising a stopper being configured to deliver a medicine from a medicine container;
a first piezoelectric actuator comprising a shaft, the first piezoelectric actuator being configured to generate a reciprocating movement of the shaft for rotating the piston at least unidirectionally; and
a clutch in contact with the piston,
the clutch being configured to translate the rotational movement of the piston into a translational movement of the piston in a forward direction for delivering the medicine from the medicine container, and
the clutch being configured to enable a backward movement of the piston in case a force applied to the piston in a backward direction exceeds a predefined value.

2. The apparatus according to claim 1, further comprising:
a wheel connected to the piston configured to translate a movement of the wheel into a movement of the piston,
the shaft being configured to rotate the piston via the wheel.

3. The apparatus according to claim 2, wherein the first piezoelectric actuator is configured to generate a non-linear reciprocating movement of the shaft for contacting the wheel during one movement of the reciprocating movement of the shaft.

4. The apparatus according to claim 2, wherein the first piezoelectric actuator further comprises a first resetting element configured to produce a formfitting contact between a tip of the shaft and the wheel.

5. The apparatus according to claim 4, wherein the tip of the shaft comprises a dented shape.

6. The apparatus according to claim 1, wherein the clutch is formed as a slipping clutch.

7. The apparatus according to claim 1, wherein the clutch comprises at least two pins configured to guide the piston.

8. The apparatus according to claim 7, wherein each pin comprises:
a movable pinion having a thread corresponding to a thread of the piston, and
a blocking element configured to prevent a movement of the pinion such that the rotational movement of the piston is translated into a translational movement of the piston in forward direction.

9. The apparatus according to claim 8, wherein the clutch is configured to enable a movement of the pinion such that a backward movement of the piston is generated in case the applied force exceeds the predefined value.

10. The apparatus according to claim 8, wherein
each pin comprises a second resetting element having a restoring force, and
wherein the predefined value depends on the restoring force of the second resetting element.

11. The apparatus according to claim 10, wherein the second resetting element is formed as a spring.

12. The apparatus according to claim 1, wherein the apparatus comprises a medicine delivery opening for delivering the medicine, wherein the medicine delivery opening corresponds to an opening of a refill package.

13. The apparatus according to claim 1, further comprising at least one further piezoelectric actuator.

14. The apparatus according to claim 1, wherein the apparatus is an insulin injector or an infusion device.

15. A method comprising:
generating a reciprocating movement of a shaft by a first piezoelectric actuator;
rotating a piston by the reciprocating movement of the shaft;
translating the rotational movement of the piston into a translational movement of the piston in a forward direction for delivering a medicine from a medicine container; and
refilling the medicine container by moving the piston backward in case a force applied to the piston in a backward direction exceeds a predefined value.
